(19) United States
(12) Patent Application Publication
(10) Pub. No.: US 2023/0367443 A1
(43) Pub. Date: Nov. 16, 2023

(54) MEDICAL DEVICE AND METHOD FOR CONTROLLING AN INPUT DEVICE

(71) Applicant: GE Precision Healthcare LLC, Wauwatosa, WI (US)

(72) Inventors: Gang Liu, Wuxi (CN); Ke Tao, Wuxi (CN); Zhiwen Wang, Wuxi (CN); Minyu Gu, Wuxi (CN); Feng Wu, Wuxi (CN)

(21) Appl. No.: 18/318,591

(22) Filed: May 16, 2023

(30) Foreign Application Priority Data
May 16, 2022 (CN) 202210527616.9

(51) Int. Cl. G06F 3/04812; G06T 7/70; G06T 7/00; G06F 3/03

(52) U.S. Cl. CPC G06F 3/04812; G06T 7/70; G06T 7/0012; G06F 3/03; G06T 2207/20081; G06T 2207/30096; G06T 2207/20084

(57) ABSTRACT

Provided in the present application are a medical device, including an input device, and a method for controlling the input device. The method includes acquiring one or more image frames with the medical device and determining a region of interest from the one or more image frames. The method includes adjusting at least one of an input action of the input device or a state of the input device according to a position of the region of interest.

### CROSS-REFERENCE TO RELATED APPLICATION

This application claims priority to Chinese patent application number 202210527616.9, filed on May 16, 2022, the entirety of which is incorporated herein by reference.

### TECHNICAL FIELD

Embodiments of the present application relate to the technical field of medical devices, and in particular to a medical device, and an input device control method and apparatus therefor.

### BACKGROUND

Currently, user input devices are commonly used in medical devices, and are indispensable peripherals used to transmit operational requirements of users to the medical devices and cause the medical devices to perform corresponding actions.

For example, a commonly used input device for an ultrasound device is a trackball, and the trackball includes a base and a ball, wherein the base is fixed and the ball can be rolled on the base. The doctor toggles (rolls) the trackball, and by reading the rolling direction or speed of the trackball, actions such as moving a cursor, selecting a menu, and performing tracing and progress bar scrolling on a display of the ultrasound device can be performed.

It should be noted that the above description of the background is only for the convenience of clearly and completely describing the technical solutions of the present application, and for the convenience of understanding for those skilled in the art.

### SUMMARY

Provided in the embodiments of the present application are a medical device, and an input device control method and apparatus therefor.

According to an aspect of an embodiment of the present application, an input device control method is provided, and the control method comprises: determining a region of interest from one or more image frames acquired by the medical device; and controlling an input action of the input device and/or a state of the input device according to the position of the region of interest, or controlling an input action of the input device and/or a state of the input device according to the position of the region of interest and a medically relevant parameter of the region of interest.

According to an aspect of an embodiment of the present application, an input device control apparatus is provided, and the control apparatus comprises: a determination unit, configured to determine a region of interest from one or more image frames acquired by the medical device; and a control unit, configured to control an input action of the input device and/or a state of the input device according to the position of the region of interest, or to control an input action of the input device and/or a state of the input device according to the position of the region of interest and a medically relevant parameter of the region of interest.

According to an aspect of an embodiment of the present application, a medical device is provided, the medical device comprising an input device and the input device control apparatus.

One of the benefits of the embodiments of the present application is that a region of interest is determined from an image frame, and the input action of the input device and/or the state of the input device are controlled according to the position of the region of interest and/or a medically relevant parameter of the region of interest, thereby reducing the time spent on viewing a scanned video, and increasing the precision and accuracy of locating the region of interest, so that the input device can be controlled in a more flexible, intelligent, and convenient manner.

With reference to the following description and accompanying drawings, specific implementations of the embodiments of the present application are disclosed in detail, and the manners in which the principle of the embodiments of the present application is employed are illustrated. It should be understood that the implementations of the present application are not thereby limited in scope. Within the spirit and scope of the appended claims, the implementations of the present application comprise various changes, modifications, and equivalents.

### BRIEF DESCRIPTION OF THE DRAWINGS

The accompanying drawings are included to provide further understanding of the embodiments of the present application, constitute a part of the specification, and are used to illustrate implementations of the present application and set forth the principles of the present application together with textual description. Obviously, the accompanying drawings in the following description are merely some embodiments of the present application, and a person of ordinary skill in the art might obtain other implementations according to the accompanying drawings without the exercise of inventive effort. In the accompanying drawings:

FIG. 1 is a schematic diagram of an input device control method according to an embodiment of the present application.

FIG. 2 is a schematic diagram of an input device control method according to an embodiment of the present application.

FIG. 3 is a schematic diagram of an input device control apparatus according to an embodiment of the present application.

FIG. 4 is a schematic diagram of a control device for an input device according to an embodiment of the present application.

FIG. 5 is a schematic diagram of a two-stage neural network model for determining regions of interest and medically relevant parameters according to an embodiment of the present application.

FIG. 6 is a schematic diagram showing a method for controlling a trackball in a playback scenario according to an embodiment of the present application.

### DETAILED DESCRIPTION

The foregoing and other features of the embodiments of the present application will become apparent from the following description with reference to the accompanying drawings. In the description and the accompanying drawings, specific implementations of the present application are specifically disclosed, and part of the implementations in which the principles of the embodiments of the present application may be employed are indicated. It should be understood that the present application is not limited to the described implementations. On the contrary, the embodiments of the present application include all modifications, variations, and equivalents falling within the scope of the appended claims.

In the embodiments of the present application, the terms “first,” “second,” etc. are used to distinguish different elements by name, but do not represent a spatial arrangement or temporal order, etc. of these elements, and these elements should not be limited by these terms. The term “and/or” includes any one of and all combinations of one or more associated listed terms. The terms “comprise,” “include,” “have,” etc. indicate the presence of the described features, elements, components, or assemblies, but do not exclude the presence or addition of one or more other features, elements, components, or assemblies.

In the embodiments of the present application, the singular forms “a”, “the”, etc. include plural forms, and should be broadly construed as “a type of” or “a class of” rather than limited to the meaning of “one.” Furthermore, the term “the” should be construed as including both the singular and plural forms, unless otherwise specified in the context. In addition, the term “according to” should be construed as “at least in part according to . . . ,” and the term “based on” should be construed as “at least in part based on . . . ,” unless otherwise specified in the context.

The features described and/or illustrated for one embodiment may be used in one or more other embodiments in the same or similar manner, combined with features in other embodiments, or replace features in other embodiments. The term “include/comprise” when used herein indicates the presence of features, integrated components, steps, or assemblies, but does not preclude the presence or addition of one or more other features, integrated components, steps, or assemblies.

In practical application, after a doctor uses a medical device to acquire a series of scanned images, the doctor often needs to play back the saved scanned images to find regions of interest therefrom for measurement, viewing, and subsequent diagnosis, etc. For example, during playback, the doctor needs to manually use an input device to view the scanned images acquired during the scan time frame by frame, and locate the regions of interest in the scanned images. However, such viewing method is inefficient and time-consuming. If the scanned images within the scan time are quickly viewed by increasing the sensitivity of the input device, it is easy to miss the regions of interest in the scanned images, which leads to a waste of time instead.

In response to at least one of the above technical problems, the embodiments of the present application provide a medical device, and an input device control method and apparatus therefor.

The following is a specific description of the embodiments of the present application with reference to the accompanying drawings.

The embodiments of the present application provide an input device control method, which is used in a medical device.

In some embodiments, the medical device includes at least one of a computed tomography device, an MRI device, an electron emission tomography device, a single photon emission computed tomography device, an ultrasound device, a monitoring device, and an endoscope, but the present application is not limited thereto, and the medical device may also be other devices from which medical imaging can be obtained.

In some embodiments, the medical device (e.g., an endoscope) captures, by an imaging device, an image or a video containing a subject to be examined, or the medical device applies a physical signal such as a visible light, x-ray, ultrasound, or magnetic field to a subject to be examined and obtains a signal intensity distribution fed back by the subject, so as to form an image or a video containing the subject, but the embodiments of the present application are not limited thereto. The medical device is electrically connected to a display, or the medical device has a display. The display may be used to show a graphical user interface, and the graphical user interface may include a region for displaying medical imaging (i.e., the image or video as described above) acquired by the medical device.

In some embodiments, the input device includes at least one of a trackball, a trackpad, a mouse, and a keyboard. The input device may be a device independent of the medical device or may be part of the medical device, and the present application is not limited thereto. The input device is electrically connected to the medical device. For example, by operating the input device, the user can perform actions such as moving a cursor, selecting a menu, performing tracing and progress bar scrolling, and keying in characters on the graphical user interface showed on the display.

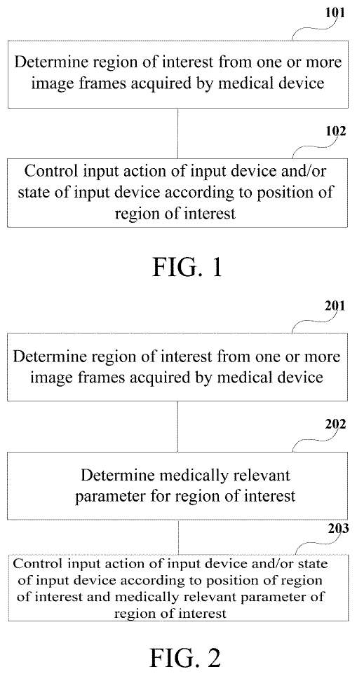

FIG. 1 is a schematic diagram of an input device control method according to an embodiment of the present application. As shown in FIG. 1, the input device control method includes: 101, determining a region of interest from one or more image frames acquired by the medical device; and 102, controlling an input action of the input device and/or a state of the input device according to the position of the region of interest.

In some embodiments, the one image frame in 101 refers to a frame in the image or video acquired by the medical device, and the plurality of image frames refer to all or a portion of the frames of the video acquired by the medical device, but the embodiments of the present application are not limited thereto. The one or more image frames may be acquired by the medical device in real time or may also be stored in the medical device after acquisition thereof.

In some embodiments, in 101, a region of interest is determined from one or more image frames, and the region of interest may be a portion of an image frame or an entire image frame, e.g., a localized portion of interest is determined as a region of interest from one image frame, or an image frame of interest is determined as a region of interest from a plurality of image frames. The region of interest may include one region or multiple regions, e.g., the region of interest may be one or more localized regions in an image frame, or one or more image frames of interest.

In some embodiments, the region of interest may be an image portion or image frame containing a target subject (e.g., an organ), or an image portion or image frame containing a lesion site, or an image portion or image frame containing a standard view, or an image portion or image frame containing a sectional view of a target, or an image portion or image frame containing an anomaly, or an image frame of optimal quality, and the embodiments of the present application are not limited thereto.

In some embodiments, in 101, the region of interest can be determined using a deep learning algorithm or a machine learning algorithm. For example, a neural network model can be used to determine the region of interest from one or more image frames, e.g., the neural network model can be a pre-trained neural network model for identifying a certain lesion (e.g., a nodule or tumor), or a pre-trained neural network model for identifying a standard view (e.g., a sectional view of the fetal head, nose, spine, arms and legs), or a pre-trained neural network model for identifying a sectional view of a target (e.g., a left-sided sectional view of the heart), etc. References can be made to the prior art for specific methods and neural network models for determining regions of interest.

In some embodiments, the neural network model processes each image frame separately, i.e., the one or more image frames are input into the neural network model sequentially, and the output result of the neural network model is a contour of a portion of interest (e.g., a lesion) in the current input image frame, as well as the width, height, area, etc. of the portion of interest. If the current input image frame contains the portion of interest, then the portion of interest can be considered as a region of interest, and the position of the region of interest in the image frame is recorded (e.g., the pixel coordinates of certain points on the contour are recorded); or, if the current input image frame contains the portion of interest, then the current input image frame is considered as a region of interest (or an image frame of interest), and the position of the region of interest in the image frame is recorded (e.g., the pixel coordinates of certain points on the contour are recorded), and the position of the region of interest (or the image frame of interest) in each image frame is recorded (e.g., the time-stamp of the image frame of interest is marked and recorded).

In some embodiments, after the position of the region of interest is determined in 101 (e.g., after the pixel coordinates of certain points on the contour and/or the time-stamp information are recorded), in 102, when the one or more image frames are being viewed (played back) or located, the input action of the input device and/or the state of the input device can be controlled according to the position of the region of interest. Controlling the input action of the input device includes: controlling the movement speed and sensitivity of the input device, wherein the movement speed of the input device can further be understood as the movement speed of a cursor, or a pointer, or a cursor in the progress bar, and the sensitivity can further be understood as the degree of change in response that corresponds to the same unit of input by the input device; and/or, controlling the input of the input device to be valid or invalid, etc. Controlling the state of the input device includes controlling the state of a vibrator, indicator light, or buzzer on the input device, etc.

Control modes for input devices in conventional methods are fixed, while in the embodiments of the present application, the input action and/or the state during viewing or locating of the position of the region of interest are different from the input action and/or the state during viewing or locating of a region other than the region of interest. In this way, the control mode of the input device can be dynamically adjusted, i.e., the input action and/or the state is dynamically adjusted, so that the input device can be controlled in a more flexible, intelligent and convenient manner.

In some embodiments, controlling the input action of the input device according to the position of the region of interest includes, during the process of viewing or locating the position of the region of interest, reducing the movement speed of the input device and/or increasing the sensitivity of the input device when the position of the region of interest is near.

For example, the medical device pre-acquires and stores a plurality of image frames (video), and when using the input device to call and play back the plurality of image frames, the conventional method is to view these images frame by frame and the movement speed and sensitivity of the input device are maintained constant, while in the embodiments of the present application, the movement speed of the input device is reduced and/or the sensitivity of the input device is increased when the position of the region of interest is near. When moving away from the position of the region of interest, the movement speed of the input device is increased and/or the sensitivity of the input device is decreased, thereby dynamically adjusting the sensitivity of the input device, so as to locate the position of the region of interest more easily when the position of the region of interest is near. The time spent on playback is reduced, thereby increasing efficiency. For example, when the input device is a trackball or a scroll wheel of a mouse, by scrolling the trackball or the scroll wheel of a mouse back and forth, the image frames are viewed forward or backward, and when the position of the region of interest is near, the movement speed of the trackball or the scroll wheel of a mouse is reduced and/or the sensitivity of the trackball or the scroll wheel of a mouse is increased, i.e., when the position of the region of interest is getting closer and closer, in correspondence with the same amount of scrolling displacement per unit (unit tooth frame), the number of frames flipped forward or backward becomes less, i.e., the movement speed becomes slower, but the sensitivity becomes higher.

For example, the medical device acquires an image frame in real time, and when a region of interest in the image frame is being located, the region of interest needs to be selected from the image frame for measurement, etc. In the conventional method, when the region of interest is selected by controlling the input device, the input device needs to be controlled such that the cursor moves on the image frame to approach and locate the region of interest, while in the embodiments of the present application, the movement speed of the input device is reduced and/or the sensitivity of the input device is increased when the position of the region of interest is near. When moving away from the position of the region of interest, the movement speed of the input device is increased and/or the sensitivity of the input device is decreased, thereby dynamically adjusting the sensitivity of the input device, so that it is easier to locate the position of the region of interest when the position of the region of interest is near. For example, when the input device is a trackball, the cursor is moved by rolling the trackball back and forth, and the movement speed of the trackball is reduced and/or the sensitivity of the input device is increased when the position of the region of interest is near, i.e., the closer the position of the region of interest is, the less the cursor movement displacement becomes in correspondence with the same scrolling displacement.

In some embodiments, controlling the input action of the input device according to the position of the region of interest includes: during the process of viewing or locating the position of the region of interest, invalidating an input of the input device or limiting a movement range of the input device on an image frame to a predetermined range when the position of the region of interest is located. Invalidating an input of the input device means that the trackball or the user can still input or conduct operations through the input device, but the signal of such input or operation is not transmitted to the medical device or display, or the medical device or display receives the signal of the input or operation but does not process the signal in a corresponding manner.

For example, the medical device pre-acquires and stores a plurality of image frames (video), and when using the input device to call and play back the plurality of image frames, the conventional method is to view the images frame by frame and the movement speed and sensitivity of the input device is maintained constant, while in the embodiments of the present application, the input from the input device is invalidated when the position of the region of interest is located, and the input device is enabled when a region other than a region of interest is located, thereby enabling a region of interest to be easily located, and allowing the region of interest to be easily viewed during an invalidation phase. For example, when the input device is a trackball or a scroll wheel of a mouse, the trackball or the scroll wheel of a mouse is scrolled back and forth to navigate forward or backward through the image frames, and the scrolling of the trackball or scroll wheel is invalidated (within a predetermined time) when the position of a region of interest is located. Such predetermined time can be determined as needed, and invalidating the movement of the trackball means that the trackball can still be scrolled (toggled), but the signal of scrolling (toggling) is not passed to the medical device or display, or the medical device or display receives the signal of scrolling (toggling) but does not process the signal in a corresponding manner.

For example, the medical device acquires an image frame in real time, and when a region of interest in the image frame is being located, the region of interest needs to be selected from the image frame for measurement, etc. In the conventional method, when selection is performed by controlling the input device, the movement range of the input device on the image frame is the entire image. In the embodiments of the present application, when the position of the region of interest is being located, the movement range of the input device on the image frame is limited to a predetermined range, so that the region of interest can be easily located. For example, when the input device is a trackball, the trackball is scrolled back and forth to move the cursor, and the movement range of the trackball on the image frame is limited to a predetermined range (the predetermined range can be slightly larger than the region of interest) when the position of the region of interest is being located.

In some embodiments, controlling the state of the input device according to the position of the region of interest includes: during the process of viewing or locating the position of the region of interest, turning on an indicator light of the input device, or causing the input device to vibrate or to produce a sound when the position of the region of interest is located.

For example, the medical device pre-acquires and stores a plurality of image frames (video), and when the plurality of image frames are called and placed back using the input device, the indicator light of the input device is turned on, or the input device is caused to vibrate or to produce a sound when the position of the region of interest is located, thereby providing a more intuitive reminder that a region of interest has been viewed or located. For example, when the input device is a trackball, the trackball is further provided with an indicator light or a vibrator, and the indicator light is caused to turn on, or the vibrator of the input device is caused to vibrate, or the buzzer of the input device is caused to produce a sound when the position of the region of interest is located, while the indicator light is not turned on, the vibrator does not vibrate or the buzzer does not produce a sound when the position of the region of interest has not been located.

For example, the medical device acquires an image frame in real time, and when the region of interest in the image frame is being located, the region of interest needs to be selected from the image frame for measurement, etc. The input device can be controlled so that the cursor moves on the image frame to approach and locate the region of interest, and when the position of the region of interest is located, the indicator light of the input device is turned on, or the input device is caused to vibrate, or the input device is caused to produce a sound, thereby providing a more intuitive reminder that a region of interest has been viewed or located. For example, when the input device is a trackball, a cursor is moved by rolling the trackball back and forth, and the indicator light is caused to turn on, or the vibrator is caused to vibrate, or the buzzer of the input device is caused to produce a sound when the cursor has moved to (has located) the region of interest, and the indicator is not turned on, the vibrator does not vibrate, or the buzzer does not produce a sound when the cursor has not moved to (has not located) the region of interest.

At least any two of the aforementioned input actions and states can be controlled simultaneously, or the actions and states can be implemented independently, and the embodiments of the present application are not limited thereto.

As can be seen from the above embodiments, controlling the input action of the input device and/or the state of the input device according to the position of the region of interest reduces the time spent on viewing the scanned video and improves the precision and accuracy of locating the region of interest, so that the input device can be controlled in a more flexible, intelligent and convenient manner.

The embodiments of the present application further provide another input device control method. Features of the input device control method in this embodiment that are the same as those of the input device control method in the preceding embodiments will not be repeated herein, and only the differences are described below. FIG. 2 is a schematic diagram of an input device control method according to an embodiment of the present application. As shown in FIG. 2, the input device control method includes: 201, determining a region of interest from one or more image frames acquired by the medical device; 202, determining a medically relevant parameter for the region of interest; and 203, controlling an input action of the input device and/or a state of the input device according to the position of the region of interest and the medically relevant parameter of the region of interest.

The implementation method of 201 is the same as that of 101, and will not be repeated herein.

In some embodiments, the medically relevant parameter for the region of interest includes an organ status and/or a lesion type. In 202, a neural network model may be used to determine the medically relevant parameter for the region of interest. For example, the region of interest identified in 201 is input into the neural network model, and the output of the neural network model may be the medically relevant parameter. The medically relevant parameter (e.g., an organ status and/or a lesion type) may be represented using a numerical score or numerical label. For example, the region of interest is a region containing a lesion, and the neural network model can score the lesion with a score ranging from 0.0 to 1.0, with higher scores indicating higher malignancy. The lesion is malignant when the score is above a threshold, and is benign when the score is below or equal to the threshold, or the lesion can be directly classified by using a classification label indicating benignness or malignancy. For example, the region of interest is the region containing a target organ (e.g., the heart), and the neural network model can classify the target organ with classification labels such as systolic, diastolic, ejection phase, and filling phase, etc.

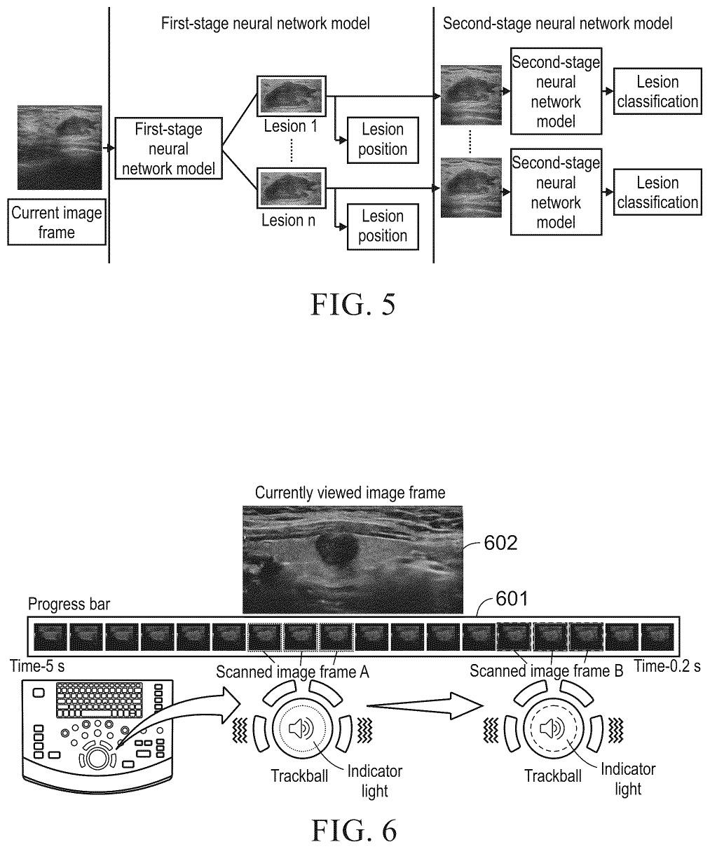

FIG. 5 is a schematic diagram of using a two-stage convolutional neural network model to obtain regions of interest and medically relevant parameters according to an embodiment of the present application. As shown in FIG. 5, the current image frame is input to the first-stage neural network model, and the region of interest 1, . . . , region of interest n in the current image frame are determined, and the width, height, area, etc. of each region of interest are also determined, and the time-stamp of the current image frame is marked and recorded. The region of interest 1, . . . , region of interest n are input into the second stage neural network model to obtain medically relevant parameters corresponding to the respective regions of interest, such as a lesion type (numerical score or numerical label).

In some embodiments, in 203, the difference from the input device control method in the preceding embodiments lies in that, in addition to the position of the region of interest, the medically relevant parameter of the region of interest also needs to be considered, and the two are combined to control the input action of the input device and/or the state of the input device. For example, the input action of the input device and/or the state of the input device can be controlled according to the position of the region of interest and the medically relevant parameter of the region of interest when one or more image frames are viewed (played back) or located, wherein controlling the input action of the input device includes controlling the movement speed and sensitivity of the input device, and/or controlling the input of the input device to be valid or invalid, etc., and controlling the state of the input device includes controlling the state of a vibrator, an indicator light, or a speaker on the input device, etc.

Control modes for input devices in conventional methods are fixed, while in the embodiments of the present application, not only the input action and/or the state during viewing or locating of the position of a region of interest are different from the input action and/or the state during viewing or locating of the position of a region other than the region of interest, but the input action and/or the state are also different when the position of the region of interest having a different medically relevant parameter is being viewed or located. Thus, the control mode of the input device can be adjusted more flexibly and dynamically. Explanations are provided below, respectively.

In some embodiments, controlling the input action of the input device according to the position of the region of interest and the medically relevant parameter of the region of interest includes, during the process of viewing or locating the position of the region of interest, reducing the movement speed of the input device and/or increasing the sensitivity of the input device when the position of the region of interest is near. References can be made to the preceding embodiments for the specific implementation method. The difference from the preceding embodiments lies in that the position of the region of interest having a different medically relevant parameter corresponds to a different reduction amount in movement speed and a different increase amount in sensitivity. For example, the movement speed of the input device is reduced to a first value and/or the sensitivity is increased to a third value during the process of viewing or locating the position of the region of interest when the position of a malignant region of interest (lesion) is near, and the movement speed of the input device is reduced to a second value and/or the sensitivity is increased to a fourth value when the position of a benign region of interest (lesion) is near, with the first value being less than the second value and the third value being greater than the fourth value. For example, the diastolic state of the heart is more useful for clinical determination of whether a certain disease is present in the heart, so during viewing of multiple image frames, the movement speed of the input device is reduced to a first value and/or the sensitivity is increased to a third value when the position of a region of interest (heart) in a diastolic state is near, and when the position of the region of interest (heart) in other states is near, the movement speed of the input device is reduced to a second value and/or the sensitivity is increased to a fourth value, with the first value being less than the second value and the third value being greater than the fourth value.

In some embodiments, controlling the input action of the input device according to the position of the region of interest and the medically relevant parameter of the region of interest includes, during the process of viewing or locating the position of the region of interest, invalidating the input of the input device when the position of the region of interest is located. References can be made to the preceding embodiments for the specific implementation method. The difference from the preceding embodiments lies in that the position of the region of interest having a different medically relevant parameter corresponds to a different invalidation time. For example, when a malignant region of interest (lesion) is located, the input of the input device is invalidated for a first period of time, and when a benign region of interest (lesion) is located, the input of the input device is invalidated for a second period of time, with the first period of time being less than the second period of time. For example, the diastolic state of the heart is more useful in clinically determining whether a certain disease is present in the heart, so during viewing of multiple image frames, the input of the input device is invalidated for a first period of time when a region of interest (heart) in a diastolic state is located, and for a second period of time when the region of interest (heart) in other states is located, with the first period of time being less than the second period of time.

In some embodiments, controlling the state of the input device according to the position of the region of interest and the medically relevant parameter of the region of interest includes, during the process of viewing or locating the position of the region of interest, turning on an indicator light of the input device, or causing the input device to vibrate, or causing the input device to produce a sound when the position of the region of interest is located. References can be made to the preceding embodiments for the specific implementation method. The difference from the preceding embodiments lies in that the position of the region of interest having a different medically relevant parameter corresponds to a different color of the indicator light, or a different vibration frequency, or a different produced sound. For example, during the process of viewing or locating the position of the region of interest, the indicator light is turned on and displayed as a first color (red) light when the position of a malignant region of interest (lesion) is located, and the indicator light is turned on and displayed as a second color (green) light when the position of a benign region of interest (lesion) is located. For example, the diastolic state of the heart is more useful for clinical determination of whether a certain disease is present in the heart, so during viewing of multiple image frames, the indicator light is turned on and displayed as a first color (red) light when the position of a region of interest (heart) in a diastolic state is located, and the indicator light is turned on and displayed as a second color (green) light when the position of a region of interest (heart) in other states is located, with the first color being different from the second color. For example, during the process of viewing or locating the position of the region of interest, the vibrator is caused to vibrate, or the buzzer is caused to produce a sound with a vibration or buzzer frequency being a first frequency when the position of a malignant region of interest (lesion) is located, and the vibrator is caused to vibrate, or the buzzer is caused to produce a sound with a vibration or buzzer frequency being a second frequency when the position of a benign region of interest (lesion) is located. For example, the diastolic state of the heart is more useful for clinical determination of whether a certain disease is present in the heart, so during viewing of multiple image frames, the vibrator is caused to vibrate, or the buzzer is caused to produce a sound with the vibration or buzzer frequency being a first frequency when the position of the region of interest (heart) in a diastolic state is located, and the vibrator is caused to vibrate, or the buzzer is caused to produce a sound with the vibration or buzzer frequency being a second frequency when the position of the region of interest (heart) in other states is located, with the first frequency being different from the second frequency.

At least any two of the aforementioned input actions and states can be controlled simultaneously, or the actions and states can be implemented independently, and the embodiments of the present application are not limited thereto.

As can be seen from the above embodiments, controlling the input action of the input device and/or the state of the input device according to the position of the region of interest and the medically relevant parameter of the region of interest reduces the time spent on viewing the scanned video, and improves the precision and accuracy of locating the region of interest, so that the input device can be controlled in a more flexible, intelligent and convenient manner, thereby making it more intuitive, quick and convenient for the user to determine regions of interest having different medically relevant parameters.

The above embodiments merely provide illustrative descriptions of the embodiments of the present application. However, the present application is not limited thereto, and appropriate variations may be made on the basis of the above embodiments. For example, each of the above embodiments may be used independently, or one or more of the above embodiments may be combined.

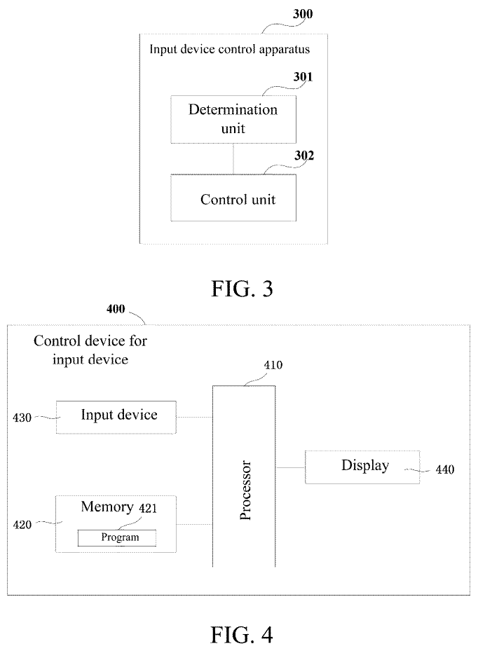

The embodiments of the present application further provide an input device control apparatus, and the input control apparatus is applied to a medical device. For the implementation method of the input device and the medical device, please refer to the preceding embodiments. FIG. 3 is a schematic diagram of an input device control apparatus according to an embodiment of the present application. As shown in FIG. 3, the control apparatus includes: a determination unit 301 configured to determine a region of interest from one or more image frames acquired by the medical device; and a control unit 302 configured to control an input action of the input device and/or a state of the input device according to the position of the region of interest, or to control an input action of the input device and/or a state of the input device according to the position of the region of interest and a medically relevant parameter of the region of interest.

In some embodiments, the determination unit 301 determines the region of interest from one or more image frames using a deep learning algorithm or a machine learning algorithm; or, the determination unit 301 may also be used to determine the medically relevant parameter for the region of interest using a deep learning algorithm or a machine learning algorithm.

In some embodiments, the input action and/or the state controlled by the control unit 302 when the position of the region of interest is being viewed or located are different from the input action and/or the state controlled by the control unit 302 when the position of a region other than the region of interest is being viewed or located.

In some embodiments, the input action and/or the state controlled by the control unit 302 are different when the position of the region of interest having a different medically relevant parameter is being viewed or located.

In some embodiments, during the process of viewing or locating the position of the region of interest, the control unit 302 reduces the movement speed of the input device and/or increases the sensitivity of the input device when the position of the region of interest is near; or, the control unit 302 invalidates an input of the input device or limits a movement range of the input device on an image frame to a predetermined range when the position of the region of interest is located; or, the control unit 302 controls an indicator light of the input device to turn on, or causes the input device to vibrate, or cause the input device to produce a sound when the position of the region of interest is located.

In some embodiments, during the process of viewing or locating the position of the region of interest, when the position of the region of interest is near or is located, the control unit 302 controls the position of the region of interest having a different medically relevant parameter to correspond to a different color of the indicator light, or a different vibration frequency, or a different produced sound, or a different movement speed and a different sensitivity.

In some embodiments, references can be made to 101-102 or 201-203 in the preceding embodiments for specific implementation methods of the determination unit 301 and the control unit 302, and the repeated contents will not be provided herein.

As can be seen from the above embodiments, controlling the input action of the input device and/or the state of the input device according to the position of the region of interest reduces the time spent on viewing the scanned video and improves the accuracy and precision of locating the region of interest, so that the input device can be controlled in a more flexible, intelligent and convenient manner. In addition, controlling the input action of the input device and/or the state of the input device in combination with the medically relevant parameter of the region of interest makes it more intuitive, quick and convenient for the user to determine regions of interest having different medically relevant parameters.

Embodiments of the present application further provide a control device for an input device. For the implementation method of the input device and the medical device, please refer to the preceding embodiments. FIG. 4 is a schematic diagram of a control device for an input device according to an embodiment of the present application. As shown in FIG. 4, a control device 400 for an input device may include: one or more processors (e.g., a central processing unit CPU) 410 and one or more memories 420 coupled to the processors 410. The memory 420 may store image frames and neural network models, etc., and in addition, a control program 421 of the input device is further provided, and is executed under the control of the processor 410. The memory 420 may include, for example, a ROM, a floppy disk, a hard disk, an optical disk, a magneto-optical disk, a CD-ROM, or a non-volatile memory card.

In some embodiments, functions of an input device control apparatus 300 are integrated into and implemented by the processor 410. The processor 410 is configured to implement the input device control method as described in the preceding embodiments.

In some embodiments, the input device control apparatus 300 is configured separately from the processor 410. For example, the input device control apparatus 300 can be configured as a chip connected to the processor 410, and the functions of the input device control apparatus 300 are implemented through the control of the processor 410.

For example, the processor 410 is configured to perform controls as follows: controlling the input action of the input device and/or the state of the input device according to the position of the region of interest, or controlling the input action of the input device and/or the state of the input device according to the position of the region of interest and a medically relevant parameter of the region of interest.

In some embodiments, for the implementation method of processor 410, references may be made to the preceding embodiments, which will not be repeated herein.

In addition, as shown in FIG. 4, the control device 400 for an input device may further include: an input device 430 and a display 440 (used to show a user graphical interface, as well as various data, image frames or parameters generated during data acquisition and processing), etc. The functions of the above components are similar to the prior art, which will not be repeated herein. It should be noted that the control device 400 for an input device does not have to include all the components shown in FIG. 4. Furthermore, the control device 400 for an input device can further include components not shown in FIG. 4, and references can be made to the related technology.

The processor 410 can respond to the operation of the input device to communicate with the medical device, display, etc., and also control the input action and/or state of the input device. The processor 410 may also be referred to as a microcontroller unit (MCU), microprocessor or microcontroller or other processor device and/or logic device, and the processor 410 may include reset circuitry, clock circuitry, chips, microcontrollers, etc. The functions of the processor 410 may be integrated into the motherboard of the medical device (e.g., the processor 410 is configured as a chip connected to the processor (CPU) of the motherboard) or may be configured independently of the motherboard, and the embodiments of the present application are not limited thereto.

For the sake of simplicity, FIG. 3 and FIG. 4 only exemplarily illustrate a connection relationship or signal flow direction between various components or modules, but it should be clear to those skilled in the art that various related technologies such as bus connection can be employed. The various components or modules can be implemented by means of a hardware facility such as a processor, a memory, etc. The embodiments of the present application are not limited thereto.

As can be seen from the above embodiments, controlling the input action of the input device and/or the state of the input device according to the position of the region of interest reduces the time spent on viewing the scanned video and improves the accuracy and precision of locating the region of interest, so that the input device can be controlled in a more flexible, intelligent and convenient manner. In addition, controlling the input action of the input device and/or the state of the input device in combination with the medically relevant parameter of the region of interest makes it more intuitive, quick and convenient for the user to determine regions of interest having different medically relevant parameters.

Embodiments of the present application further provide a medical device that includes an input device and the input device control apparatus 300 as described in the preceding embodiments. The implementation methods of the medical device, the input device, and the input device control apparatus 300 are as described above, which will not be repeated herein.

In some embodiments, the medical device further includes a display, or the medical device is electrically connected to a display which can show a graphical user interface. The graphical user interface may include an area for displaying medical imaging (images or video as described above) acquired by the medical device. For example, the display can show the current image frame acquired by the medical device in real time, or a plurality of previously acquired image frames (video) stored in the medical device can also be called for browsing and display.

In some embodiments, when an image frame is shown on the display and the image frame contains a region of interest, the image frame corresponding to the region of interest (i.e., the image frame containing the region of interest) is marked using a bounding box of a predetermined color. When multiple image frames are shown on the display, the multiple image frames can be shown as thumbnails in a progress bar, while simultaneously showing the currently viewed image frame, and the image frame corresponding to the region of interest (i.e., the image frame of interest) in the progress bar is marked using a bounding box of a predetermined color. As such, users can view or locate the region of interest in a more intuitive, quick and convenient manner.

In some embodiments, image frames corresponding to regions of interest having different medically relevant parameters are marked using bounding boxes of different colors.

For example, when an image frame is shown on the display and the image frame contains a malignant region of interest, a bounding box of a first color is used for marking the image frame corresponding to the region of interest. When the image frame contains a benign region of interest, a bounding box of a second color is used for marking the image frame corresponding to the region of interest. When a plurality of image frames are shown on the display, the plurality of image frames can be shown as thumbnails in a progress bar, while simultaneously showing the currently viewed image frame. The image frame corresponding to the malignant region of interest in the progress bar is marked using a bounding box of a first color. The image frame corresponding to a benign region of interest is marked using a bounding box of a second color. The first color is different from the second color.

For example, when an image frame is shown on the display and the image frame contains a region of interest (heart) in a diastolic state, a bounding box of a first color is used for marking the image frame corresponding to the region of interest. When the image frame contains the region of interest (heart) in other states, a bounding box of a second color is used for marking the image frame corresponding to the region of interest. When multiple image frames are shown on the display, the multiple image frames can be shown as thumbnails in a progress bar, while simultaneously showing the currently viewed image frame. The image frame corresponding to the region of interest (heart) in a diastolic state is marked using a bounding box of a first color in the progress bar. The image frames corresponding to the region of interest (heart) in the other states are marked using a bounding box of a second color. The first color is different from the second color.

As such, users can view or locate regions of interest having different medically relevant parameters in a more intuitive, quick and convenient manner.

In some embodiments, the medical device may further include a chassis assembly (not shown in the figure) that is used to process data (e.g., to obtain medical imaging), and the processed data can be sent and shown on a display, and the chassis assembly includes electronic components such as a motherboard, chips, diodes, capacitors, resistors, etc.

The functions of the input device control apparatus may be integrated in a processor, microcontroller unit (MCU), microprocessor or microcontroller or other processor device and/or logic device. The functions of the processor may be integrated into the motherboard of the medical device (e.g., configuring the processor as a chip connected to the motherboard processor (CPU)) or may be configured independently of the motherboard, and the embodiments of the present application are not limited thereto.

In some embodiments, the medical device may further include other components, and references can be made to the prior art for the specifics, which will not be repeated herein.

In some embodiments, the input device control apparatus generates a control signal for the input device according to the position of the region of interest, or the position and the medically relevant parameter. For example, the control signal includes a signal to control the input action of the input device and/or the state of the input device. The motherboard of the medical device receives the input signal from the input device and generates a processing signal according to the control signal and the input signal. The processing signal is a signal that includes the processing of actions such as cursor movement, menu selection, tracing, progress bar scrolling, character typing, etc. on the graphical user interface shown on the display.

The following is an example in which the medical device is an ultrasound device and the input device is a trackball. An illustrative explanation of the input device control method according to the embodiments of the present application is provided in a playback scenario. It will be understood by those skilled in the art that the embodiments of the present application may also be applicable to other medical devices as described above, or to other input devices, or to other scenarios.

The ultrasound device is used to perform ultrasound examination to diagnose diseases. The ultrasound device includes a display, a chassis assembly, an ultrasound probe, and an input device. The input device includes a keyboard, a button and a trackball. The ultrasound probe is used to receive and transmit sound waves, and transmit the acquired reflection data to the chassis assembly. The chassis assembly processes the reflection data, and the processed data can be stored in a storage device of the chassis assembly and shown on the display.

For example, FIG. 6 is a schematic diagram showing a method for controlling a trackball in an ultrasound device according to an embodiment of the present application. As shown in FIG. 6, when using the ultrasound probe to scan the subject to be examined, by clicking the button on the input device 603, all the scanned image frames within 5 s forward or 5 s backward of the currently scanned image frames can be saved, and a scanned video with a length of 5 s will be obtained after the completion of the scan. Using a neural network model, a scanned image frame A containing a benign lesion and a scanned image frame B containing a malignant lesion in the scanned video are determined, and during playback of the 5 s scanned video, a plurality of scanned image frames can be shown in a progress bar 601 as thumbnails on a display, while simultaneously showing the currently viewed scanned image frame 602, wherein the scanned image frame A is marked using a bounding box of a first color, and the scanned image frame B is marked using a bounding box of a second color. When scrolling through the trackball and viewing the scanned image frames forward or backward, the movement speed of the trackball is reduced and/or the sensitivity of the input device is increased when the scanned image frame A and the scanned image frame B are near. When the currently viewed image frames are (by means of locating) the scanned image frame A and the scanned image frame B, the input of the trackball is caused to be invalid, and at the same time, the indicator light of the trackball is turned on, and the trackball is caused to vibrate and make a beeping sound. In this case, the colors of the trackball indicator light are different when the scanned image frame A and the scanned image frame B are located.

In some embodiments, optionally, after the region of interest (image frame of interest) is determined, an automatic viewing mode can further be selected via the input device, which automatically plays the image frames of interest on the display. For example, after determining the scanned image frame A containing a benign lesion and the scanned image frame B containing a malignant lesion in the scanned video, if the automatic viewing mode is selected, the scanned image frame A and the scanned image frame B are displayed sequentially, with each scanned image frame being displayed for n seconds, wherein n is a positive number that can be determined as needed.

As can be seen from the above embodiments, controlling the input action of the input device and/or the state of the input device according to the position of the region of interest reduces the time spent on viewing the scanned video and improves the accuracy and precision of locating the region of interest, so that the input device can be controlled in a more flexible, intelligent and convenient manner. In addition, controlling the input action of the input device and/or the state of the input device in combination with the medically relevant parameter of the region of interest makes it more intuitive, quick and convenient for the user to determine regions of interest having different medically relevant parameters.

The embodiments of the present application further provide a computer readable program, wherein upon execution of the program, the program causes a computer to perform, in an apparatus or medical device, the input device control method described in the preceding embodiments.

The embodiments of the present application further provide a storage medium storing a computer readable program, wherein the computer readable program causes a computer to perform, in an apparatus or medical device, the input device control method described in the preceding embodiments.

The above embodiments merely provide illustrative descriptions of the embodiments of the present application. However, the present application is not limited thereto, and appropriate variations may be made on the basis of the above embodiments. For example, each of the above embodiments may be used independently, or one or more of the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims aim to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A method for controlling an input device, where the input device is configured to be applied to a medical device, the method comprising:
acquiring one or more image frames with the medical device;
determining a region of interest from the one or more image frames; and
adjusting at least one of an input action of the input device or a state of the input device according to a position of the region of interest.

2. The method of claim 1, wherein said adjusting at least one of the input action of the input device or the state of the input device comprises adjusting the input action of the input device.

3. The method of claim 2, wherein said adjusting the input action of the input device comprise at least one of adjusting a movement speed of the input device according to the position of the region of interest or adjusting a sensitivity of the input device according to the position of the region of interest.

4. The method of claim 3, wherein said adjusting the input action of the input device comprises at least one of reducing the movement speed of the input device or increasing the sensitivity of the input device when the region of interest in near.

5. The method of claim 4, wherein said adjusting the input action of the input device comprises at least one of increasing the movement speed of the input device or decreasing the sensitivity of the input device when moving away from the position of the region of interest.

6. The method of claim 1, wherein said acquiring the one or more image frame comprises acquiring a plurality of image frames and wherein the position of the region of interest is with respect to the one of the plurality of image frames being viewed.

7. The method of claim 1, wherein the position of the region of interest is with respect to a cursor in one of the one or more image frame, and wherein the cursor is controlled by the input device.

8. The method of claim 1, wherein said adjusting at least one of the input action of the input device or the state of the input device comprises adjusting the state of the input device, and wherein said controlling the state comprises performing one or more of the following when the region of interest is located: turning on an indicator light of the input device to display a color, causing the input device to vibrate, or causing the input device to produce a sound.

9. The method of claim 8, further comprising controlling one or more of a color of the indicator light, a vibration frequency, or a sound based on a medically relevant parameter of the position of the region of interest.

10. The method of claim 1, wherein said determining the region of interest comprises determining the region of interest from the one or more image frames using a deep learning algorithm or a machine learning algorithm.

11. A medical device comprising:
an input device; and
an input device control apparatus for controlling the input device, and implemented by a processor, the input device control apparatus comprising:
a determination unit configured to determine a region of interest in one or more image frames; and
a control unit configured to adjust at least one of an input action of the input device or a state of the input device according to a position of the region of interest.

12. The medical device of claim 11, wherein the control unit is configured to adjust the input action of the input device.

13. The medical device of claim 12, wherein the control unit is configured to adjust the input action of the input device by at least one of adjusting a movement speed of the input device according to the position of the region of interest or adjusting a sensitivity of the input device according to the position of the region of interest.

14. The medical device of claim 13, wherein the control unit is configured to adjust the input action of the input device by at least one of reducing the movement speed of the input device or increasing the sensitivity of the input device when the position of the region of interest is near.

15. The medical device of claim 14, wherein the control unit is configured to adjust the input action of the input device by at least one of increasing the movement speed of the input device or decreasing the sensitivity of the input device when moving away from the position of the region of interest.

16. The medical device of claim 11, wherein the one or more image frames comprises a plurality of image frames, and wherein the position of the region of interest is with respect to one of the plurality of image frames being viewed.

17. The medical device of claim 11, wherein the position of the region of interest is with respect to a cursor in one of the one or more image frames.

18. The medical device of claim 11, wherein the control unit is configured to adjust the state of the input device by performing one or more of the following when the region of interest is located: turning on an indicator light of the input device to display a color, causing the input device to vibrate, or causing the input device to produce a sound.

19. The medical device of claim 18, wherein the control unit is further configured to control one or more of a color of the indicator light, a vibration frequency, or a sound based on a medically relevant parameter of the position of the region of interest.

20. The medical device of claim 11, wherein the determination unit is configured to determine the region of interest from the one or more image frames using a deep learning algorithm or a machine learning algorithm.
